(19) United States
(12) Patent Application Publication
(10) Pub. No.: US 2024/0426829 A1
(43) Pub. Date: Dec. 26, 2024

(54) COMPOSITIONS AND METHODS FOR THE DETECTION OF BRAIN INJURY

(71) Applicants: The Children's Hospital of Philadelphia, Philadelphia, PA (US); Monell Chemical Senses Center, Philadelphia, PA (US)

(72) Inventors: Akiva Cohen, Bala Cynwyd, PA (US); Kristy Arbogast, Media, PA (US); Bruce A. Kimball, Jenkintown, PA (US); Gary K. Beauchamp, Philadelphia, PA (US); Christina L. Master, Philadelphia, PA (US); Young Eun Lee, Philadelphia, PA (US)

(21) Appl. No.: 18/750,367

(22) Filed: Jun. 21, 2024

Related U.S. Application Data
(60) Provisional application No. 63/522,785, filed on Jun. 23, 2023.

(51) Int. Cl. G01N 33/64; A61B 10/00

(52) U.S. Cl. CPC G01N 33/64; A61B 10/007; A61B 2560/0431; G01N 2800/28

(57) ABSTRACT

Compositions and methods are provided for the detection and/or diagnosis of traumatic brain injury.

This application claims priority under 35 U.S.C. § 119 (e) to U.S. Provisional Patent Application No. 63/522,785, filed on Jun. 23, 2023. The foregoing application is incorporated by reference herein.

### FIELD OF THE INVENTION

The present invention relates to the detection and diagnosis of a traumatic brain injury, particularly a mild traumatic brain injury or concussion.

### BACKGROUND OF THE INVENTION

Traumatic brain injury (TBI) afflicts up to two million people annually in the United States, and is the primary cause of death and disability in young adults and children. TBI often causes enduring disabilities including emotional alterations, cognitive impairment and memory dysfunction.

TBI is presently diagnosed by a physician and there are many instances, whether it be on the athletic or battlefield, where concussions go undiagnosed. Recent studies have explored advanced neuroimaging and blood biomarkers. However, neither approach is applicable for widespread use. Currently, there is no biomarker or group of biomarkers that can be used to diagnose a concussion or traumatic brain injury. The lack of an objective biomarker(s) hampers mild traumatic brain injury (mTBI) diagnosis and limits the ability to monitor patients and evaluate interventions. A biomarker(s) for traumatic brain injury is desirable in order to rapidly and accurately diagnose a concussion/mild traumatic brain injury and to ensure rapid medical treatment, which is important for a more effective and successful therapy.

### SUMMARY OF THE INVENTION

In accordance with the instant invention, methods for detecting and/or diagnosing in a subject a traumatic brain injury are provided. In certain embodiments, the method comprises detecting and/or measuring the amount of one or more biomarkers of TBI in a biological sample obtained from the subject. In certain embodiments, the method comprises detecting and/or measuring the amount of one or more biomarkers of TBI in the gaseous or vapor phase of the biological sample. In certain embodiments, the biological sample is a fluid (e.g., urine). In certain embodiments, the biological sample is obtained from the subject within days, particularly within 48 hours (e.g., within 24 hours, 12 hours, or 1 hour), of the purported traumatic brain injury. In certain embodiments, the traumatic brain injury is mild or moderate. In certain embodiments, the traumatic brain injury is a concussion.

In certain embodiments, the biomarkers of TBI comprise one or more small molecule biomarkers. In certain embodiments, the biomarker is an aliphatic ketone. In certain embodiments, the biomarkers comprise at least 3-methyl-4-hexen-2-one. In certain embodiments, the biomarkers comprise at least phenylacetone. In certain embodiments, the biomarkers comprise at least 3-octen-2-one, 3-nonen-2-one, and/or 3-decen-2-one. In certain embodiments, the biomarkers comprise at least 3-octen-2-one.

In certain embodiment, the biomarker(s) is measured and/or detected by gas chromatography (e.g., headspace gas chromatography), liquid chromatography, mass spectrometry, gas chromatography/mass spectrometry (e.g., headspace gas chromatography/mass spectrometry), ion mobility spectrometry (e.g., field asymmetric ion mobility spectrometry), absorption spectroscopy, electronic noses, optical methods, ionization methods, and/or trained animal detection (e.g., biosensor animal. In certain embodiments, the biomarker(s) is measured and/or detected by chemical analysis, such as gas chromatography/mass spectrometry. In certain embodiments, the biomarker(s) is measured and/or detected by a biosensor (e.g., dipstick or test strip).

In accordance with another aspect of the instant invention, devices for detecting and/or measuring one or more TBI biomarkers (e.g., in a biological sample or in the gaseous or vapor phase) are provided. In certain embodiments, the device is configured and/or programmed to detect and/or measure one or more biomarkers of the instant invention.

### BRIEF DESCRIPTIONS OF THE DRAWING

FIG. 1 provides group average normalized peak heights for gas chromatograms from sham (square) and injured (circle) mice. An early increase (1 hour and 12 hours) in the values for injured mice compared to sham mice was observed compared to sham. Peak heights are normalized to the peak height for the carvone standard.

FIG. 2 provides a bar graph of diagnostic ketones identified in urine collected from 16 concussed youths (Day 0=same day; n=11; Day 1=one day post injury; n=5) and 8 healthy youths. For each ketone, the bars are healthy control, Day 0, and Day 1, from left to right. * indicates Day 0>control.

### DETAILED DESCRIPTION OF THE INVENTION

A significant problem with diagnosing TBI, particularly mild to moderate TBI, is the lack of non-invasive biomarkers. Currently, the best tool for a non-invasive assessment of TBI is an assessment of symptoms and a physical exam performed by a medical professional. However, to be effective, this tool can require a priori established individual baseline measures, which may be unavailable or difficult to obtain. Thus, establishing non-invasive biomarkers that can be identified and assessed, even in the field, following suspected TBI inducing trauma would be of great benefit. The biomarkers will also allow for the monitoring of recovery from the TBI.

Urine has a high potential as a valuable source for a biomarker as it is easily collected, especially for children, and can be distinguished by odor volatiles. Proteomic and metabolomic analyses of urine identified differences in concussed athletes compared to healthy controls (Daisy, et al. (2022) Neurology 98 (2): e186-e198; Wanner, et al. (2021) Frontiers in Neurology, 12:645829). Further, discriminatory volatile metabolites have been identified that appear 48 hours after injury in urine of mice with mild lateral fluid percussion injury (mLFPI) and persisting up to two weeks after injury (Kimball, et al. (2016) Chemical Senses 41:407-414). A biomarker that can be assessed more closely to the time of injury is desirable for quicker treatment of the subject.

Herein, it is demonstrated that brain injured subjects have an altered urine profile compared to uninjured, normal subjects during the acute period of injury (e.g., <48 hours, <36 hours, <24 hours, etc.). In certain embodiments, the altered urine profile is detectable within hours (e.g., 1 hour) of the brain injury. Further, the chemical profile of urine from a subject having experienced a brain injury can be chemically distinguished from an uninjured or sham-treated subject.

In accordance with the instant invention, methods of detecting and/or diagnosing a traumatic brain injury in a subject are provided. As used herein, “traumatic brain injury” or “TBI” refers to an acquired brain injury or a head injury, when a trauma causes damage to the brain. Trauma includes, e.g., post-head trauma, impact trauma, and other traumas to the head such as, for example, traumas caused by accidents and/or sports injuries, traumas incurred on the battlefield (e.g., explosions, bombs, explosive devices, etc.), concussive injuries, penetrating head wounds, brain tumors, stroke, heart attack, meningitis, viral encephalitis, and other conditions that deprive the brain of oxygen. In certain embodiments, the trauma is an external, physical force or blow, particularly to the head. In certain embodiments, the traumatic brain injury is a mild traumatic brain injury. In certain embodiments, the traumatic brain injury is a concussion.

In certain embodiments, the subject is a youth, adolescent, or young adult. In certain embodiment, the subject is less than about 40 years old or less than about 35 years old. In certain embodiments, the subject is less than 25 years old, less than 21 years old, or less than 18 years old. In certain embodiments, the subject is more than about 10 years old.

The damage to the brain can be focal (confined to one area of the brain) or diffuse (involving more than one area of the brain). Clinically, traumatic brain injury can be rated as mild, moderate or severe based on TBI variables that include duration of loss of consciousness (LOC), Glasgow Coma Score (GCS; e.g., mild 13-15; moderate=9-12; severe=≤8) and post traumatic amnesia (see, e.g., Levin et al. (1979) J. Nervous Mental Dis., 167:675-84; Holm et al. (2005) J. Rehabil. Med., 37:137-41). In certain embodiments, the TBI detected and/or diagnosed by the instant invention is mild or moderate, particularly mild.

In certain embodiments, the traumatic brain injury can be repetitive, where the brain is subject to repeated physical loading to the brain. Generally, repetitive traumatic brain injury is typically a mild to moderate form of closed brain injury repeatedly suffered by a subject (e.g., athlete (e.g., football player), soldier, etc.), resulting in increased incidence of impaired motor, cognitive, and/or behavioral impairments months to years following the traumatic brain injuring events. Individuals subjected to such repetitive brain injury appear to have increased susceptibility to certain neurological disorders, such as Alzheimer's disease, chronic traumatic encephalopathy (CTE), and/or Parkinson's Disease.

In certain embodiments, the traumatic brain injury can result from a closed head injury. The closed head injury may be transient or prolonged. A “closed head injury” refers to a brain injury when the head suddenly and violently rotated or accelerated but an object does not break through the skull. In some embodiments, the closed head injury is a concussion or contusion. A concussion is often a mild form of traumatic brain injury resulting in temporary impairment of neurological function, and where there are generally no gross structural changes to the brain as the result of the condition. A contusion is a distinct area of swollen brain tissue mixed with blood released from broken blood vessels. A contusion can also occur in response to shaking of the brain back and forth within the confines of the skull, an injury referred to as “contrecoup.” As used herein, a closed head injury refers to an injury due to an external, physical trauma and does not encompass brain injury resulting from “internal” forces such as ischemia/reperfusion and stroke.

In certain embodiments, the methods of detecting and/or diagnosing a traumatic brain injury in a subject of the instant invention comprise detecting and/or measuring one or more biomarkers in a biological sample from the subject. In certain embodiments, the biological sample is a fluid. In certain embodiments, the biological sample is breadth. In certain embodiments, the biological fluid is blood, sweat, tears, urine, saliva, or other non-blood fluid. In certain embodiments, the biological fluid is urine. The methods may further comprise obtaining the biological sample from the subject.

The methods may also comprise comparing the biomarker(s) (e.g., presence and/or amount of one or more biomarker) of the subject with the biomarker(s) in the same biological sample (e.g., fluid) from a subject without a TBI (i.e., a normal or healthy subject) and/or comparing the biomarker(s) of the subject with the biomarker(s) in the same biological sample (e.g., fluid) from a subject with a TBI. In other words, it is determined whether the biomarker(s) (e.g., presence and/or amount of one or more biomarker) from the subject corresponds to/matches the biomarker(s) (e.g., presence and/or amount of one or more biomarker) from a subject without TBI (thereby indicating no TBI in the test subject) or corresponds to/matches the biomarker(s) (e.g., presence and/or amount of one or more biomarker) from a subject with TBI (thereby indicating a TBI in the test subject). The methods of the instant invention may comprise actively performing the analysis on the positive and/or negative control samples or may comprise comparison to previously performed experiments (e.g., standards or references). In certain embodiments, the methods comprise comparing the biomarker(s) (e.g., presence and/or amount of one or more biomarker) of the subject with the biomarker(s) in the same biological sample (e.g., fluid) from the subject prior to the TBI.

The methods of the instant invention may further comprise treating a subjected determined to have had a TBI with a TBI therapeutic. In certain embodiment, the TBI therapeutic is a branched chain amino acid (e.g., valine, leucine, and/or isoleucine), such as a solution (e.g., oral) comprising one or more branched chain amino acids (see, e.g., U.S. Patent Application Publication No. 20160058726, incorporated herein by reference). Other examples of TBI therapeutics include without limitation: those provided in Tani et al. (Pharmaceuticals (2022) 15 (7): 838, particularly those in Table 1, incorporated herein by reference), ghrelin (OXE-103; Shao, et al., Front. Neurosci. (2018) 12:445), and amantadine (Reddy et al., J. Head Trauma Rehabil. (2013) 28 (4): 260-5). In certain embodiments, an analgesic, pain reliever, and/or headache medication may be administered to the subject. Examples of analgesics, pain relievers, and/or headache medication include, without limitation, a non-steroidal anti-inflammatory drug (NSAID; e.g., ibuprofen, naproxen, celecoxib, meloxicam, and/or aspirin) or acetaminophen.

Branched chain amino acids (BCAAs) are amino acids that have a fork or branch in the side chain. BCAAs include leucine, isoleucine and valine and precursors or analogs thereof. BCAAs may be administered in their free forms or salts thereof, as dipeptides, tripeptides, polypeptides (e.g., from about 2 to about 10 amino acids), and/or BCAA-rich proteins (e.g., proteins comprising at least 25%, at least 50%, or at least 75% or more BCAA content). In certain embodiments, dipeptides, tripeptides and polypeptides may include two or more BCAAs. Where non-BCAAs are included in a dipeptide, tripeptide, or polypeptide, the non-BCAAs may be any amino acid, particularly alanine and/or glycine. Examples of dipeptides include, without limitation: isoleucyl-leucine, leucyl-alanine, alanyl-leucine, alanyl-isoleucine, alanyl-valine, glycyl-leucine, glycyl-isoleucine, and glycyl-valine.

The BCAAs may be administered to the subject by any means. In certain embodiments, the BCAAs are administered orally or in a liquid consumable (e.g., a palatable/drinkable composition (see, e.g., U.S. Patent Application Publication No. 20160058726, incorporated herein by reference)). The BCAAs administered to the subject may include one, two, or all three of valine, leucine, and isoleucine. In certain embodiments, valine, leucine, and isoleucine are administered in approximately equivalent amounts (e.g., a 1:1:1 ratio or +5%). In certain embodiments, only one or two of valine, leucine, and isoleucine are administered (e.g., valine may be administered). The amount of BCAA may be determined, for example, by weight or molar amount. While the BCAAs will often be administered in equivalent amounts, excess amounts of one or two of the BCAAs may be administered. For example, up to ten times, up to five times, up to three times, or up to two times excess of one or two amino acids compared to another may be administered. For example, excess valine compared to leucine and/or isoleucine may be administered.

Leucine precursors, such as pyruvate, and metabolites, such as β-hydroxy-β-methylbutyrate and α-ketoisocaproate, exhibit properties similar to those of leucine. These compounds may be administered as BCAAs as they are converted into the above-mentioned BCAA in vivo.

In certain embodiments, at least about 40 g, at least about 45 g, at least about 50 g, at least about 54 g, at least about 60 g, at least about 70 g, at least about 75 g, at least about 80 g, at least about 85 g, at least about 90 g, at least about 95 g, at least about 100 g, or more of BCAAs are administered to the subject per day. In certain embodiments, about 40 g to about 100 g of BCAAs are administered daily, particularly about 50 g to about 100 g, about 60 g to about 100 g, about 60 g to about 75 g, or about 60 g. Taking the subject's weight into account, at least about 40 g/70 kg, at least about 50 g/70 kg, at least about 60 g/70 kg, at least about 70 g/70 kg, or more of BCAAs are administered to the subject per day. In certain embodiments, about 40 g/70 kg to about 100 g/70 kg of BCAAs are administered daily, particularly about 50 g/70 kg to about 100 g/70 kg, about 60 g/70 kg to about 100 g/70 kg, about 60 g/70 kg to about 75 g/70 kg, or about 60 g/70 kg. The BCAAs may be administered in more than one dosage to reach the daily goal (e.g., administered twice, three times, four times or more daily).

In certain embodiments, the subject is administered at least 1000 g, at least 1250 g, at least 1500 g, at least 1750 g, at least 2000 g, at least 2250 g, at least 2500 g, at least 2750 g, or at least 3000 g of BCAAs during the treatment period. In certain embodiments, the treatment period is at least 7 days, at least 10 days, or at least two weeks. In certain embodiments, the treatment period is two weeks or less, three weeks or less, four weeks or less, or five weeks or less. In certain embodiments, the BCAAs are administered evenly or nearly evenly across the treatment period (e.g., with the same or similar daily dose). In certain embodiments, the BCAAs are administered on consecutive days (e.g., daily).

In certain embodiments, the BCAAs are administered immediately or soon after the traumatic brain injury event and/or diagnosis by the methods of the instant invention. For example, the BCAAs may be administered at least within a month of injury and/or diagnosis, within two weeks of injury and/or diagnosis, within about the first 2, 3, 4 or 7 days after injury and/or diagnosis, within about the first day after injury and/or diagnosis, or within about the first hour after injury and/or diagnosis. In certain embodiments, the subject is administered BCAA therapy within 72 hours, within 48 hours, or within 24 hours of the traumatic brain injury and/or diagnosis. In certain embodiments, the BCAAs are administered within about the first 2 days of the injury and/or diagnosis. The BCAAs may be administered continually (e.g., every day) after the injury and/or diagnosis for at least one week, particularly at least two weeks, at least three weeks, at least four weeks or more.

The BCAAs of the instant invention may also be administered with acetate. Acetate may be administered as acetic acid or a pharmaceutically acceptable salt thereof, such as calcium acetate. In certain embodiments, the acetate is present in a hydrophobic form, such as glyceryl triacetate (GTA; the acetate triester of glycerol). In certain embodiments, about 0.5 mg/kg to about 100 mg/kg of GTA are administered daily, particularly about 0.5 mg/kg to about 10 mg/kg, about 2.5 mg/kg to about 7.5 mg/kg, or about 5 mg/kg.

In certain embodiments, the biological sample is obtained from the subject within about 3 weeks after the purported TBI event. In certain embodiments, the biological sample is obtained from the subject within about 3 weeks, 2, weeks, 1 week, 6 days, 5 days, 4 days, 3 days, 2 days, 1 day, or less after the purported TBI event. In certain embodiments, the biological sample is obtained from the subject within about 48 hours after the purported TBI event. In certain embodiments, the biological sample is obtained within about 36 hours, about 24 hours, about 22 hours, about 20 hours, about 18 hours, about 16 hours, about 14 hours, about 12 hours, about 10 hours, about 8 hours, about 6 hours, about 4 hours, about 2 hours, or about 1 hour after the purported TBI event. Inasmuch as the biomarkers of the biological sample may be volatile, the biological sample should be handled to avoid loss of volatile chemicals (e.g., maintained in a sealed container, optionally, frozen). Alternatively, the sample may be tested immediately for the presence of the biomarker(s) to avoid loss of volatile chemicals.

The methods of the instant invention can be performed at more than one timepoint after the purported TBI (e.g., a timecourse). By taking multiple assessments, the recovery from the TBI and/or efficacy of a treatment can also be monitored (e.g., by determining if the chemical signature returns to normal). In accordance with the instant, methods of determining the efficacy of a treatment against TBI are provided, wherein the method comprises determining if the chemical signature of a subject having a TBI and administered the therapy returns to normal faster than a subject having a TBI without therapy. In certain embodiments, the method comprises administering a therapy to a subject and detecting and/or measuring one or more biomarkers in a biological sample from the subject at a later time point, optionally more than once. The method may further comprise detecting and/or measuring one or more biomarkers in a biological sample from the subject before and/or at the time of the administration of the therapy (e.g., as a baseline).

In certain embodiments, the biomarker(s) of the biological sample is detected and/or measured by chemical analysis (e.g., a chemometric approach). In certain embodiments, the biomarker(s) of the biological sample is detected and/or measured in the gaseous or vapor phase of the biological sample. The chemical make-up of the biological sample or presence and/or amount of TBI biomarkers in the biological sample can be determined by any method known in the art. Examples of methods for detecting and/or measuring the biomarker(s) of the instant invention include, without limitation: gas chromatography (e.g., headspace gas chromatography), liquid chromatography, mass spectrometry, gas chromatography/mass spectrometry (e.g., headspace gas chromatography/mass spectrometry), ion mobility spectrometry (e.g., field asymmetric ion mobility spectrometry), absorption spectroscopy, electronic noses, optical methods, ionization methods, and/or trained animal detection (e.g., biosensor animal). In certain embodiments, the chemical analysis is gas chromatography. In certain embodiments, the chemical analysis is gas chromatography/mass spectrometry. In certain embodiments, the chemical analysis is headspace gas chromatography/mass spectrometry. In certain embodiments, the chemical analysis is ion mobility spectrometry (e.g., field asymmetric ion mobility spectrometry). In certain embodiments, the biomarkers are detected and/or measured by a volatile organic compounds (VOC) analyzer. In certain embodiments, the chemical analysis is an optical method. In certain embodiments, the chemical analysis is amperometric. In certain embodiments, the chemical analysis is colorimetric. In certain embodiments, the chemical analysis is performed to detect and determine the amount (either relatively or quantitatively) of molecules other than proteins. In certain embodiments, the chemical analysis is performed to detect and determine the amount (either relatively or quantitatively) of small molecules. The data can be analyzed using a univariate or multiple variate analysis to allow for the classification of sample based on a few (e.g., 1, 2, 3, 4, 5, or more) peaks or components.

In certain embodiments, the biomarker(s) of the biological sample is detected and/or measured by a field, portable and/or handheld device. In certain embodiments, the biomarker(s) of the biological sample is detected and/or measured in the gaseous or vapor phase of the biological sample. In certain embodiments, the biomarker(s) of the biological sample is detected and/or measured by a field, portable and/or handheld version of a device which performs the detection method (e.g., gas chromatograph (e.g., headspace gas chromatograph), liquid chromatograph, mass spectrometer, gas chromatograph/mass spectrometer (e.g., headspace gas chromatograph/mass spectrometer), ion mobility spectrometer (e.g., field asymmetric ion mobility spectrometer), absorption spectrometer, electronic noses, etc.). In certain embodiments, the biomarker(s) is detected and/or measured by a volatile organic compounds (VOC) analyzer, particularly a field, portable, and/or handheld VOC analyzer. In certain embodiments, the biomarker(s) is detected and/or measured by a gas chromatograph, particularly a field, portable, and/or handheld gas chromatograph. In certain embodiments, the biomarker(s) is detected and/or measured by a gas chromatograph/mass spectrometer, particularly a field, portable, and/or handheld gas chromatograph/mass spectrometer. In certain embodiments, the biomarker(s) is detected and/or measured by an ion mobility spectrometer, particularly a field, portable, and/or handheld ion mobility spectrometer (e.g., field asymmetric ion mobility spectrometer). In certain embodiments, the biomarker(s) is detected and/or measured by optical methods, particularly a field, portable, and/or handheld optical device. In certain embodiments, the biomarker(s) is detected and/or measured by ionization methods, particularly a field, portable, and/or handheld ionization device. In certain embodiments, the field, portable and/or handheld device is amperometric. In certain embodiments, the field, portable and/or handheld device is colorimetric.

In certain embodiments, the biomarker(s) of the biological sample is detected by a biosensor. The method may comprise exposing the biosensor to the test biological sample. In certain embodiments, the biosensor is a dipstick or test strip. In certain embodiments, the dipstick or test strip comprises absorbent material. In certain embodiments, the biosensor is an electrochemical sensor. In certain embodiments, the biosensor comprises compounds which specifically bind the biomarker(s). In certain embodiments, the biosensor will provide or emit a detectable signal (e.g., generated upon binding to a biomarker) to indicate the presence or absence of the target analyte (biomarker). In certain embodiments, the biosensor will allow for rapid detection of the presence or absence and/or amount of a target analyte (e.g., biomarker) in a sample.

The instant invention also encompasses methods of detecting and/or measuring TBI biomarkers in a biological sample. In certain embodiments, the biomarker(s) is detected and/or measured in the gaseous or vapor phase of the biological sample. In certain embodiments, the biological sample is a fluid. In certain embodiments, the biological sample is breath. In certain embodiments, the biological fluid is blood, sweat, urine, tears, saliva, or other non-blood fluid. In certain embodiments, the biological fluid is urine. The methods may further comprise obtaining the biological sample from the subject.

In certain embodiments, the biomarkers of the instant invention for any of the methods described herein include at least one of phenylacetone, 3-methyl-4-hexen-2-one, 4-hepten-2-one, 5-hepten-2-one, antioxidant derivative 1, 2-acetyl-2-thiazole, o-Toluidine, 2,3-dehydro-exo-brevicomin (DHB), butyl ether, and 6-methyl-3-heptanone. In certain embodiments, the biomarkers of the instant invention for any of the methods described herein include at least one of phenylacetone, 3-methyl-4-hexen-2-one, 4-hepten-2-one, 5-hepten-2-one, antioxidant derivative 1, 2-acetyl-2-thiazole, butyl ether, and 6-methyl-3-heptanone. In certain embodiments, the methods of the instant invention use 1, 2, 3, 4, 5, 6, 7, 8, 9, or all 10 of the biomarkers. In certain embodiments, the biomarkers comprise at least 3-methyl-4-hexen-2-one. In certain embodiments, the biomarkers comprise at least phenylacetone. In certain embodiments, the biomarkers comprise at least 3-methyl-4-hexen-2-one and phenylacetone. In certain embodiments, the biomarkers comprise at least 3-methyl-4-hexen-2-one and phenylacetone as well as any 1, 2, or 3 of 4-hepten-2-one, 5-hepten-2-one, and 6-methyl-3-heptanone.

In certain embodiments, the biomarkers of the instant invention for any of the methods described herein include at least one of 3-octen-2-one, 3-nonen-2-one, and 3-decen-2-one. In certain embodiments, the methods of the instant invention use 1, 2, or all 3 of the biomarkers. In certain embodiments, the biomarkers comprise at least 3-octen-2-one. In certain embodiments, the biomarkers comprise at least 3-nonen-2-one. In certain embodiments, the biomarkers comprise at least 3-decen-2-one.

In certain embodiments, the biomarkers of the instant include at least one of 3-octen-2-one, 3-nonen-2-one, and 3-decen-2-one and 1, 2, 3, 4, 5, 6, 7, 8, 9, or all 10 of phenylacetone, 3-methyl-4-hexen-2-one, 4-hepten-2-one, 5-hepten-2-one, antioxidant derivative 1, 2-acetyl-2-thiazole, o-Toluidine, 2,3-dehydro-exo-brevicomin (DHB), butyl ether, and 6-methyl-3-heptanone. In certain embodiments, the biomarkers of the instant include 1) at least one of 3-octen-2-one, 3-nonen-2-one, and 3-decen-2-one and 2) 3-methyl-4-hexen-2-one and/or phenylacetone and/or 1, 2, or 3 of 4-hepten-2-one, 5-hepten-2-one, and 6-methyl-3-heptanone.

In certain embodiments, the biomarkers of the instant invention (e.g., for any of the methods and/or devices described herein) include at least one ketone, particularly an aliphatic ketone. In certain embodiments, the aliphatic ketone has four to twelve carbons, four to eleven carbons, four to ten carbons, or four to nine carbons. In certain embodiments, the aliphatic ketone has 4, 5, 6, 7, 8, 9, 10, 11, or 12 carbons, particularly 8, 9, or 10 carbons. In certain embodiments, the aliphatic ketone is linear (e.g., unbranched). In certain embodiments, the aliphatic ketone is unsaturated. In certain embodiments, the aliphatic ketone comprises a ketone (e.g., ═O) on carbon number two. In certain embodiments, the biomarker is a four to twelve, four to eleven, four to ten, or four to nine carbon ketone, wherein the ketone is on carbon two. In certain embodiments, the biomarker is a seven to eleven or eight to ten carbon ketone, particularly wherein the ketone is on carbon two. In certain embodiments, the aliphatic ketone comprises a ketone on carbon number two and is unsaturated. In certain embodiments, the aliphatic ketone comprises a ketone on carbon number two and comprises at least one double bond. In certain embodiments, the aliphatic ketone comprises a ketone on carbon number two and comprises a double bond between carbons three and four (e.g., at position 3).

In accordance with another aspect of the instant invention, devices for detecting and/or measuring one or more TBI biomarkers (e.g., in a biological sample) are provided. In certain embodiments, the device is configured and/or programmed to detect and/or measure one or more biomarkers of the instant invention. In certain embodiments, the device is a field, portable and/or handheld device. In certain embodiments, the device is configured to detect and/or measure the gaseous or vapor phase of the biological sample. In certain embodiments, the device is a gas chromatograph (e.g., headspace gas chromatograph), liquid chromatograph, mass spectrometer, gas chromatograph/mass spectrometer (e.g., headspace gas chromatograph/mass spectrometer), ion mobility spectrometer (e.g., field asymmetric ion mobility spectrometer), absorption spectrometer, or electronic nose. In certain embodiments, the device is a volatile organic compounds (VOC) analyzer, particularly a field, portable, and/or handheld VOC analyzer. In certain embodiments, the device is a gas chromatograph, particularly a field, portable, and/or handheld gas chromatograph. In certain embodiments, the device is a gas chromatograph/mass spectrometer, particularly a field, portable, and/or handheld gas chromatograph/mass spectrometer. In certain embodiments, the device is an ion mobility spectrometer, particularly a field, portable, and/or handheld ion mobility spectrometer (e.g., field asymmetric ion mobility spectrometer). In certain embodiments, the device is an optical device, particularly a field, portable, and/or handheld optical device. In certain embodiments, the device is amperometric. In certain embodiments, the device is colorimetric. In certain embodiments, the device is configured and/or programmed to provide or emit a detectable signal (e.g., color, light, readout, etc.) to indicate the presence, absence, and/or amount of the target analyte (biomarker). In certain embodiments, the device is or comprises a biosensor. In certain embodiments, the biosensor is a dipstick or test strip. In certain embodiments, the biosensor comprises compounds which specifically bind the biomarker(s). In certain embodiments, the biosensor provides or emits a detectable signal (e.g., generated upon binding to a biomarker) to indicate the presence or absence of the target analyte (biomarker). In certain embodiments, the biosensor allows for rapid detection of the presence or absence and/or amount of a target analyte (e.g., biomarker) in a sample.

### Definitions

The singular forms “a,” “an,” and “the” include plural referents unless the context clearly dictates otherwise.

As used herein, the term “subject” refers to an animal, particularly a mammal, particularly a human.

As used herein, “cognitive impairment” refers to an acquired deficit in at least one of the following: memory function, problem solving, orientation, and abstraction. The deficiency typically impinges on an individual's ability to function independently.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g., chromatographic methods, agarose or polyacrylamide gel electrophoresis, HPLC analysis, and the like).

The terms “isolated” is not meant to exclude artificial or synthetic mixtures with other compounds or materials, or the presence of impurities that do not interfere with the fundamental activity, and that may be present, for example, due to incomplete purification, or the addition of stabilizers.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, preservative, antioxidant, solubilizer, emulsifier, adjuvant, excipient, bulking substances, auxilliary agent or vehicle with which an active agent of the present invention is administered. Pharmaceutically acceptable carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described, for example, in “Remington's Pharmaceutical Sciences” by E. W. Martin.

The term “pathology” refers to any deviation from a healthy or normal condition, such as a disease, disorder, syndrome, or any abnormal medical condition.

The term “treat” as used herein refers to any type of treatment that imparts a benefit to a patient suffering from an injury (e.g., TBI), including improvement in the condition of the patient (e.g., in one or more symptoms), delay in the progression of the condition, etc.

As used herein, the term “prevent” refers to the prophylactic treatment of a subject who is at risk of developing a condition and/or sustaining an injury (e.g., TBI) resulting in a decrease in the probability that the subject will develop conditions associated with the injury.

A “therapeutically effective amount” of a compound or a pharmaceutical composition refers to an amount effective to prevent, inhibit, or treat a particular injury and/or the symptoms thereof. For example, “therapeutically effective amount” may refer to an amount sufficient to modulate the pathology associated traumatic brain injury in a patient.

As used herein, the term “subject” refers to an animal, particularly a mammal, particularly a human.

As used herein, “diagnose” refers to detecting and identifying a disease in a subject. The term may also encompass assessing, evaluating, and/or prognosing the disease status (progression, regression, stabilization, response to treatment, etc.) in a patient known to have the disease.

As used herein, the term “prognosis” refers to providing information regarding the impact of the presence of a disease or disorder (e.g., TBI) on a subject's future health (e.g., expected morbidity or mortality, the likelihood of developing a disease or disorder (e.g., cognitive impairment), and the severity of the disease or disorder). In other words, the term “prognosis” refers to providing a prediction of the probable course and outcome of the disease or the likelihood of recovery from the disease or disorder.

As used herein, the term “small molecule” refers to a substance or compound that has a relatively low molecular weight (e.g., less than 4,000, less than 2,000, particularly less than 1 kDa or 800 Da). Typically, small molecules are organic, but are not proteins, polypeptides, or nucleic acids, though they may be amino acids or dipeptides.

As used herein, the term “aliphatic” refers to hydrocarbon groups that are not aromatic. Aliphatic groups encompass saturated hydrocarbons and unsaturated hydrocarbons, including those that are linear or branched. Aliphatic groups include alkyl, alkenyl, and alkynyl groups. In certain embodiments, the term “aliphatic” refers to a straight or branched, saturated or unsaturated hydrocarbon having less than 30 carbons, particularly four to fourteen carbons.

The following examples describe illustrative methods of practicing the instant invention and are not intended to limit the scope of the invention in any way.

### EXAMPLE 1

Mild traumatic brain injury (mTBI), or concussion, is exceedingly common but the current state of diagnosis is primarily clinical, relying on subjective report of symptoms, presenting significant challenges for both diagnosis and treatment. Physical examination deficits, cognitive impairment, and neurobehavioral alterations supplement the clinical diagnosis, but there is to date no universally deployed diagnostic biomarker or endophenotype for concussive neurodysfunction (Zemek, et al. (2016) JAMA 315 (10): 1014; McCrory, et al. (2017) Br. J. Sports Med., 51 (11): 838-847). More recently, there has been exploration of advanced neuroimaging as a more sensitive and objective measure of neurological deficits associated with injury. However, imaging does not have the potential for rapid and widespread use in diagnostic screening as it is too costly and requires specialized healthcare settings (McCrea, et al. (2017) Br. J. Sports Med., 51 (12): 919-929).

A promising approach involves discovery of objective body fluid biomarkers that could be used to diagnose patients and subsequently monitor their recovery and response to treatment and therapy. The most commonly investigated sources for prospective biomarkers are proteins or protein fragments present in blood due to blood brain barrier breakdown caused by mTBI. Several potential candidates have been identified but are not practical for regular sideline or training room assessment and, thus, have yet to be implemented for diagnosis or monitoring of recovery during treatment (Siman, et al. (2013) Front. Neurol., 4:190; Zetterberg, et al. (2013) Nature Reviews Neurology 9:201-210; Mannix, et al. (2020) J. Neurotrauma, 37 (19): 2029-2044; Mayer, et al. (2018) Neurosci. Biobehav. Reviews 94:149-165). In contrast, urine is easy and requires no specialized training to collect and allows for sample storage at room temperature while awaiting analysis. Proteomic analysis of urine identified several biomarkers that were downregulated in concussed college athletes compared to healthy controls (Daisy, et al. (2022) Neurology, 98 (2): e186-c198). In a study of male youth athletes, the urinary metabolome was analyzed via 1H NMR spectroscopy identifying a set of metabolites that was effective at classifying those with sport related concussion (Wanner, et al. (2021) Front. Neurol., 12:645829). 2-hydroxybutyrate and lactose were the metabolites with the most promise.

Urine can be characterized by body odor volatiles (volatile metabolites originating from bodily fluids) which have been shown to be altered in numerous disease states. Volatile metabolomic approaches can be used for human studies of autoimmune diseases, cancers (Bajtarevic, et al. (2009) BMC Cancer 9:348; Bernabei, et al. (2008) Sensors and Actuators β-Chemical 131:1-4; Jezierski, et al. (2015) J. Breath Res., 9:027001), tuberculosis (Syhre, et al. (2009) Tuberculosis, 89 (4): 263-6; Mahoney, et al. (2013) Psychological Record 63:21-26; Mgode, et al. (2012) Tuberculosis 92:535-542), gastrointestinal disorders (Probert, et al. (2009) J. Gastrointestin. Liver Dis., 18 (3): 337-43) and diabetes (Greiter, et al. (2010) Diabetes Tech. Therap., 12:455-463). Chemometric evaluations of these conditions can reveal the presence of a novel volatile metabolite in patient samples (Phillips, et al. (2012) Tuberculosis 92:314-320; van Oort, et al. (2018) J. Breath Res., 12 (2): 024001) or a pattern of changes in multiple previously established metabolites (Amann, et al. (2014) J. Breath Res., 8:034001; Kimball, B.A. (2016) Bioanalysis 8:1987-1991; Kimball, et al. (2016) Chemical Senses 41:407-414; Kimball, et al. (2016) Scientific Reports 6:19495).

Specific to TBI, trained biosensor mice can discriminate between urine from mice subjected to mild lateral fluid percussion injury (mLFPI) and appropriate surgical sham controls, on the basis of volatile urinary metabolites detected at 48 hours after mild TBI and present up to 15 days post-injury (Kimball, B.A. (2016) Bioanalysis 8:1987-1991; Kimball, et al. (2016) Chemical Senses 41:407-414; Kimball, et al. (2016) Scientific Reports 6:19495). Five specific urinary volatiles (exo-brevicomin, α-Farnesene, o-toluidine, dimethyl sulfone, and formanilide) were altered by brain injury (Kimball, et al. (2016) Scientific Reports 6:19495). The post-injury period is extraordinarily dynamic marked by transient hypermetabolism shifting to protracted hypometabolism, and progression of inflammation from restorative to deleterious. Extending the analysis to the acute time period (<48 hours) is critical to establishing the pre-clinical scientific foundation that will provide support for eventual translation of the urine biomarker for clinical use. Therefore, in the current study, pristine urine was collected at 1, 12, 24, 48 and 96 hours after mLFPI or sham treatment directly from the bladders of mice and analyzed them by gas chromatography coupled with mass spectrometry (GC/MS) to determine if an earlier biomarker of injury might be present for rapid diagnoses of mTBI or concussion.

### Methods

### Mouse Fluid Percussion Brain Injury

Mild Lateral fluid percussion injury (LFPI) was performed as described (Kimball, B.A. (2016) Bioanalysis 8:1987-1991; Cole, et al. (2010) PNAS 107:366-371; Witgen, et al. (2005) Neuroscience 133:1-15). Briefly, C57BL/6 mice were anesthetized with a combination of ketamine (2.6 mg/kg) and xylazine (0.16 mg/kg) and without breaching the dura a 3 mm craniectomy was conducted over the right parietal area between bregma and lambda in the anterior-posterior direction, and the sagittal suture and lateral cranial ridge in the medial-lateral direction. A Luer-loc needle hub was secured to the skull over the opening with cyanoacrylate and dental acrylic, the hub was capped, the scalp was sutured closed, and the animal was allowed to recover. The following day, the animal was anesthetized with isoflurane (2% oxygen at 500 ml/minute). The needle hub was filled with isotonic sterile saline and connected via saline-filled tubing to the LFPI device, consisting of a large fluid-filled cylinder with a piston opposite the mouse connection end, and a pendulum to strike the piston and generate the fluid pressure pulse. The animal was placed on its left side and the pendulum was raised to a pre-determined height and released to deliver a pressure pulse of 1.8-2.0 atm. The fluid line was then disconnected, and the mouse righting time was recorded. Righting time has been shown to correlate with the injury severity (Morehead, et al. (1994) J. Neurotrauma 11:657-667), and a righting time of 250-350 seconds was used to represent mild injury (Witgen, et al. (2005) Neuroscience 133:1-15). Furthermore, studies at this injury severity have demonstrated little to no cell death, no cavitation or tissue loss supporting the mild nature of the injury (Witgen, et al. (2005) Neuroscience 133:1-15). The animal was re-anesthetized with isoflurane for scalp closure. Sham animals received all the above except the fluid pressure pulse. Fifty male mice received mLFPI treatment and an additional 50 received an equivalent sham treatment.

### Urine Collection

Urine was collected directly from the bladder of the mice at predetermined times ranging from 1 to 96 hours post-injury (Table 1). Mice were maintained under isoflurane anesthesia and a vertical incision was made on the lower abdomen (taking care not to damage the intestines). The bladder (normally found just above the penis) was identified by its translucent mass, slightly darker than the surrounding tissue. Curved forceps were used to slightly lift the bladder out of the body cavity, being careful not to put pressure on the bladder itself. As the bladder rested on the forceps, it was carefully punctured with a sterile 1 ml TB syringe (27G×½) to draw the urine into the syringe. Individual urine samples were delivered to sterile glass vials and the mouse was euthanized by decerebration. Samples were stored at −80° C. prior to being transferred for storage at −30° C. prior to headspace analyses.

TABLE 1

| Time (hr) | mLFPI | Sham |
|---|---|---|
| 1 | 10 (8) | 10 (9) |
| 12 | 10 (8) | 10 (8) |
| 24 | 10 (8) | 10 (10) |
| 48 | 10 (8) | 10 (8) |
| 96 | 10 (9) | 10 (10) |

Number of male C57BL/6 mice in each of the injured and sham cohorts, subject to urine collection at 1-, 12-, 24-, 48-, or 96-hours post-surgery. The number in parentheses indicates data availability for statistical evaluation.

### Headspace GC/MS

50 μL of urine was placed in a 20-mL headspace vial and fortified with 10 μL of an internal standard consisting of 70 μg/mL L-carvone in water (such that 700 ng was delivered to each sample). Multiple quality control (QC) samples (to monitor chromatographic system suitability) consisting of empty vials or vials containing only 700 ng of L-carvone were distributed throughout each chromatographic run. Quality control samples were also used to assist in exclusion of chromatographic peaks not related to urine. Samples were analyzed using a HT3™ dynamic headspace analyzer (Teledyne Tekmar) with a Supelco™ Trap K Vocarb™ 3000 thermal desorb trap (Sigma-Aldrich) attached to a Trace™ Ultra gas chromatograph (Thermo Scientific) equipped with a single quadrupole mass spectrometer (ISQ™, Thermo Scientific). Sample vials were incubated at 40° C. and swept with helium for 10 minutes at 75 mL/minute as volatile metabolites were collected on the thermal trap. Following collection, the trap was heated to 260° C. and volatiles desorbed directly onto the gas chromatograph equipped with 30 m×0.25 μm Stabilwax™-DA (Restek) capillary column. Split injections (5:1) were made with a column flow of 2.0 mL/minute and a split vent flow of 10.0 mL/minute. The starting GC oven temperature was 40° C. (3 minutes) and ramped to 260° C. at a rate of 7° C./minute. The mass spectrometer was operated in scan mode from 33-400 m/z following a five-minute solvent delay necessitated by the presence of ethanol cosolvent in the internal standard solution. Tentative chromatographic peak identifications were based on the NIST Standard Reference Database.

### Chemometric Analyses

Chromatographic data were exported to MetAlign® software for peak alignment and noise elimination (Lommen, A. (2009) Analytical Chemistry 81:3079-3086). Resulting data were processed by MSClust for mass spectral extraction and assignment of a single peak response value based on the extracted selected ion monitoring (SIM) trace (Tikunov, et al. (2012) Metabolomics 8:714-718). Chromatographic peak responses were normalized to the L-carvone response from each sample. Determination of the absolute concentration of a particular volatile requires comparison to an external standard for that volatile. Such standards were not available for all 68 compounds, therefore “concentration” comparisons here are made only between chromatographic peaks for the same compound. Chromatographic peaks that were present in urine samples and not present in quality control samples were considered to be of urine origin. Data were subjected to principal components analysis (PCA) using Unscrambler® (CAMO Software; Oslo, Norway) to visually identify outliers exhibiting undue influence or leverage in residual plots.

### Statistical Analyses

A multivariate analysis of variance (MANOVA) was performed with the L-carvone standard normalized gas chromatographic peak responses. Treatment (LFPI or sham), time (1, 12, 24, 48, 96 hour), and the interaction were fixed effects. The multiple different peak responses in the chromatograph were termed “volatiles” for the MANOVA. For univariate tests (treatment, time, and the interaction), the false discovery rate controlling procedure was used to account for conducting 68 univariate tests of individual volatiles (Benjamini, et al. (1995) J. Royal Statistical Soc. Series β-Methodological 57:289-300). Comparisons of means (post-hoc linear contrasts) were also conducted for the significant volatiles to determine if a) 1 hour LFPI responses were significantly greater than 1 hour sham responses, and b) 1 hour LFPI responses were significantly greater than the average sham responses across the 96 hour study. The false discovery rate controlling procedure was similarly employed to account for multiple contrasts.

### Results

Insufficient urine volume for reliable analysis was obtained from nine of the 100 mice and therefore these samples were excluded. Data obtained from 91 chromatograms revealed that five exhibited large residual variance and/or made a disproportionate contribution to the PCA results and were also excluded. Thus, data from 86 mice representing collections at 1-, 12-, 24-, 48-, and 96-hours post-injury or sham surgery formed the analytical data set (41 sham and 45 injured) (Table 1). Sixty-eight volatile compounds were detected and subjected to multivariate and univariate ANOVA testing to assess the effect of injury status and urine collection time on concentrations (Table 2).

TABLE 2

| time | peak identification | Sham ± SE | Injured ± SE | p value | α |
|---|---|---|---|---|---|
| 5.61 | m-xylene | 0.530 ± 0.112 | 2.876 ± 2.099 | 0.0842 | 0.0250 |
| 5.62 | 3-penten-2-one | 2.332 ± 0.844 | 13.78 ± 8.605 | 0.0973 | 0.0272 |
| 5.74 | enol ether product of HMH | 5.851 ± 1.657 | 34.22 ± 25.20 | 0.1137 | 0.0287 |
| 5.97 | 3-heptanone | 0.325 ± 0.217 | 2.098 ± 1.177 | 0.0895 | 0.0265 |
| 6.11 | 1-butanol | 4.217 ± 2.822 | 24.30 ± 13.08 | 0.0685 | 0.0228 |
| 6.15 | nitromethane | 9.562 ± 2.559 | 26.25 ± 14.58 | 0.4371 | 0.0478 |
| 6.49 | o-xylene | 0.068 ± 0.073 | 0.216 ± 0.083 | 0.2670 | 0.0404 |
| 6.58 | 2-heptanone | 3.937 ± 2.566 | 26.92 ± 13.47 | 0.2514 | 0.0382 |
| 6.72 | limonene | 0.005 ± 0.002 | 0.021 ± 0.012 | 0.1665 | 0.0338 |
| 7.12 | *6-methyl-3-heptanone | 0.600 ± 0.112 | 2.471 ± 0.988 | 0.0060 | 0.0074 |
| 7.20 | 3-methylcyclopentanone | 1.035 ± 0.283 | 2.968 ± 1.067 | 0.0421 | 0.0176 |
| 7.41 | 2-penten-1-ol acetate | 1.418 ± 0.578 | 13.25 ± 6.178 | 0.0093 | 0.0088 |
| 7.44 | 2-pentyl furan | 0.019 ± 0.005 | 0.099 ± 0.042 | 0.0191 | 0.0125 |
| 7.84 | 3-methyl-3-hexen-2-one | 1.578 ± 0.650 | 6.265 ± 1.809 | 0.0537 | 0.0206 |
| 8.00 | *5-hepten-2-one | 9.898 ± 5.019 | 83.72 ± 27.37 | 0.0010 | 0.0029 |
| 8.09 | *3-methyl-4-hexen-2-one | 0.012 ± 0.005 | 0.446 ± 0.154 | <0.0001 | 7.4E−04 |
| 8.20 | o-cymene | 0.007 ± 0.002 | 0.021 ± 0.008 | 0.1251 | 0.0316 |
| 8.33 | *4-hepten-2-one | 0.217 ± 0.101 | 2.531 ± 0.789 | 0.0003 | 0.0022 |
| 8.65 | CH₂ subd. exo-brevicomin | 0.865 ± 0.422 | 2.630 ± 0.989 | 0.1366 | 0.0331 |
| 8.83 | exo-brevicomin | 0.378 ± 0.347 | 1.744 ± 0.688 | 0.0713 | 0.0243 |
| 8.93 | 3-hepten-2-one | 0.436 ± 0.130 | 1.566 ± 0.406 | 0.0124 | 0.0110 |
| 9.47 | 6-methyl-5-hepten-3-one | 2.085 ± 0.406 | 5.111 ± 1.883 | 0.0588 | 0.0213 |
| 10.38 | dimethyl trisulfide | 0.017 ± 0.006 | 0.074 ± 0.024 | 0.3137 | 0.0434 |
| 10.46 | *2-acetyl-2-thiazoline | 0.017 ± 0.010 | 0.085 ± 0.029 | 0.0018 | 0.0037 |
| 10.71 | nonanal | 0.136 ± 0.040 | 0.279 ± 0.123 | 0.3294 | 0.0441 |
| 10.78 | *butyl ether | 0.541 ± 0.255 | 1.811 ± 0.690 | 0.0046 | 0.0066 |
| 10.93 | *DHB | 1.554 ± 0.757 | 5.416 ± 2.082 | 0.0043 | 0.0059 |
| 11.95 | 2-sec-Butyl-4,5- | 0.563 ± 0.163 | 1.203 ± 0.375 | 0.0102 | 0.0096 |
| | dihydrothiazole | | | | |
| 12.45 | 2-ethyl-1-hexanol | 0.449 ± 0.177 | 1.349 ± 0.385 | 0.0228 | 0.0132 |
| 12.52 | 2-decanone | 0.012 ± 0.008 | 0.056 ± 0.020 | 0.0282 | 0.0147 |
| 12.61 | decanal | 0.011 ± 0.007 | 0.048 ± 0.017 | 0.0176 | 0.0118 |
| 13.10 | benzaldehyde | 0.780 ± 0.280 | 2.082 ± 0.982 | 0.1824 | 0.0353 |
| 13.44 | linalool | 0.058 ± 0.013 | 0.138 ± 0.046 | 0.0436 | 0.0184 |
| 13.62 | octanol | 0.005 ± 0.001 | 0.018 ± 0.008 | 0.2591 | 0.0390 |
| 13.76 | 4-cyanocyclohexene | 0.004 ± 0.001 | 0.012 ± 0.005 | 0.1161 | 0.0301 |
| 14.03 | 6-methyl-6-hydroxy-3- | 0.214 ± 0.078 | 0.931 ± 0.551 | 0.0451 | 0.0199 |
| | heptanone | | | | |
| 14.25 | 4-methyl-3-pentenoic acid | 0.015 ± 0.005 | 0.064 ± 0.016 | 0.0420 | 0.0169 |
| 14.31 | succinic acid dimethyl ester | 0.008 ± 0.002 | 0.013 ± 0.004 | 0.0848 | 0.0257 |
| 15.26 | acetophenone | 0.783 ± 0.161 | 0.628 ± 0.280 | 0.3516 | 0.0456 |
| 15.40 | methyl (ms)methyl sulfide | 0.020 ± 0.016 | 0.060 = 0.021 | 0.0332 | 0.0154 |
| 15.42 | β-farnesene | 0.004 ± 0.001 | 0.018 ± 0.007 | 0.2175 | 0.0360 |
| 15.53 | isovaleric acid | 0.018 ± 0.014 | 0.170 ± 0.112 | 0.1718 | 0.0346 |
| 16.10 | caprolactone | 0.006 ± 0.005 | 0.018 ± 0.007 | 0.3111 | 0.0419 |
| 16.48 | *phenylacetone | 0.034 ± 0.007 | 0.137 ± 0.029 | <0.0001 | 0.0015 |
| 16.78 | α-farnesene | 0.007 ± 0.010 | 0.018 ± 0.010 | 0.4671 | 0.0493 |
| 17.32 | 1-ethyl-4-isobutylbenzene | 0.003 ± 0.001 | 0.020 ± 0.011 | 0.0620 | 0.0221 |
| 17.74 | *o-toluidine | 0.079 ± 0.049 | 0.287 ± 0.069 | 0.0031 | 0.0051 |
| 18.72 | *antioxidant† | 0.068 ± 0.037 | 0.197 ± 0.062 | 0.0018 | 0.0044 |
| 19.15 | dimethyl sulfone | 0.053 ± 0.040 | 0.195 ± 0.100 | 0.3076 | 0.0412 |
| 19.30 | butylated hydroxytoluene | 0.004 ± 0.002 | 0.014 ± 0.008 | 0.4095 | 0.0471 |
| 22.12 | cedrol | 0.003 ± 0.001 | 0.012 ± 0.005 | 0.3343 | 0.0449 |
| 22.87 | formanilide | 0.004 ± 0.006 | 0.024 ± 0.009 | 0.0093 | 0.0081 |

Mass spectrometry identification of chromatogram peaks for samples collected one hour after injury or sham surgery. Of the 68 compounds detected, 54 were identified and are listed here in elution order. Asterisks and bold font denote compounds significantly different between injured and sham (based on univariate ANOVA). Alpha is the error level used to control for false discovery.
Abbreviations: DHB, 2,3-dehydro-exo-brevicomin; ds, differently substituted; HMH, 6-methyl-6-hydroxy-3-heptanone; ms, methylsulfonyl; SE, standard error; subs., substituted.
†propanoic acid, 2-methyl, 1-(1,1-dimethyl)-2-methyl-1,3-propanediyl ester, derivative of Irganox ® 1076 (Cherif Lahimer, et al. (2017) Arabian J. Chem., 10: S1938-S1954).

MANOVA indicated a significant injury-dependent increase in the concentration of a subset of the volatiles, and also a variation in volatile concentration as a function of time since injury (between-subjects effect for 15 treatment*time, F4,76=3.91, p=0.0061; within-subjects effects for volatile, F68,5168=26.47, p<0.0001 and volatile*treatment*time, F272,5168=2.88, p<0.0001). Univariate ANOVA testing indicated that ten of the 68 urine volatiles showed injury dependent differences in the peak values and their time course (Table 3).

TABLE 3

| Peak Identification | Sham ± SE | Injured ± SE | p-value | α |
|---|---|---|---|---|
| Phenylacetone | 0.034 ± 0.007 | 0.137 ± 0.029 | <0.0001 | 0.0007 |
| 3-Methyl-4-hexen- | 0.012 ± 0.005 | 0.446 ± 0.154 | <0.0001 | 0.0014 |
| 2-one | | | | |
| 4-Hepten-2-one | 0.217 ± 0.101 | 2.531 ± 0.789 | 0.0003 | 0.0022 |
| 5-Hepten-2-one | 9.898 ± 5.019 | 83.72 ± 27.37 | 0.0010 | 0.0029 |
| Antioxidant | 0.068 ± 0.03 | 0.197 ± 0.062 | 0.0018 | 0.0036 |
| derivative1 | | | | |
| 2-Acetyl-2-thiazole | 0.017 ± 0.010 | 0.085 ± 0.029 | 0.0018 | 0.0051 |
| o-Toluidine | 0.079 ± 0.049 | 0.287 ± 0.069 | 0.0031 | 0.0044 |
| 2,3-Dehydro-exo- | 1.554 ± 0.757 | 5.416 ± 2.082 | 0.0043 | 0.0058 |
| brevicomin (DHB) | | | | |
| Butyl ether | 0.541 ± 0.255 | 1.811 ± 0.690 | 0.0046 | 0.0066 |
| 6-Methyl-3- | 0.600 ± 0.112 | 2.471 ± 0.988 | 0.0060 | 0.0073 |
| heptanone | | | | |

Tentative mass spectrometry identification of the 10 chromatogram peaks significantly influenced by injury compared to sham (based on the univariate ANOVA). Peak normalized values at one hour are shown. Alpha is the error level used to control for false discovery rate.

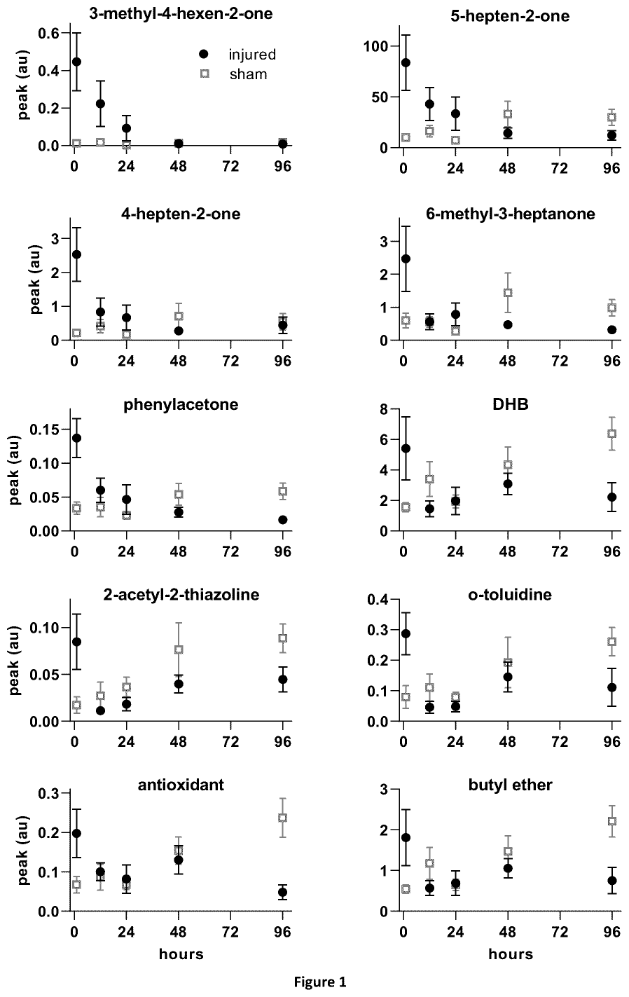

The urine concentration of these ten volatiles varied according to injury status over 96 hour and followed one of two general patterns (FIG. 1)-either an initial large elevation and return near zero, or an initial large elevation followed by a smaller sustained elevation. The novel metabolite 3-methyl-4-hexen-2-one showed the clearest difference between injured and sham—the injured peak at 1 hour was large and prominent, while the sham responses were close to zero. Using post-hoc linear contrasts, for each of the ten volatiles identified via univariate testing, the peak response at one hour was significantly greater in samples from injured mice compared to sham controls (Table 4). For samples from sham mice, the volatile concentration either remained constant or increased with time. To understand the effect of this increase on the observed treatment effect, the injured values at one hour were compared to the average of the sham values over all 96 hours. For six of the ten urine volatiles, the concentration at 1 hour for the injured mice was significantly greater than the average of sham responses across 96 hours (Table 4), indicating that the acute response of the injured cohort was still distinct from the mean sham response.

TABLE 4

| | 1 hr LFPI vs. | 1 hr LFPI vs. |
|---|---|---|
| | 1 hr Sham | Sham Avg |
| Tentative Peak Identification | p-value | p-value |
| 3-Methyl-4-hexen-2-one | <0.0001 | <0.0001 |
| Phenylacetone | <0.0001 | <0.0001 |
| 4-Hepten-2-one | <0.0001 | <0.0001 |
| 5-Hepten-2-one | <0.0001 | <0.0001 |
| 6-Methyl-3-heptanone | 0.0011 | 0.0002 |
| o-Toluidine | 0.0035 | 0.0107 |
| 2-Acetyl-2-thiazole | 0.0038 | n.s. (0.0501) |
| 2,3-Dehydro-exo-brevicomin | 0.0091 | n.s. (0.101) |
| (DHB) | | |
| Antioxidant derivative | 0.0114 | n.s. (0.0624) |
| Butyl ether | 0.0114 | n.s. (0.125) |

Comparisons of means (based on post hoc linear contrasts) for 1 hour post injury vs 1 hour post sham surgery, and 1 hour post injury vs the mean of all sham time points.

Herein, 10 volatile compounds were identified in mouse urine which were significantly elevated one hour after TBI versus sham, and six of which were elevated at one hour in injured mice versus the sham average over all time points. Nine of the ten compounds have links to TBI, further evidencing that they are reliable biomarkers. The compound 3-methyl-4-hexene-2-one showed the clearest increase after injury, and by itself, or in combination with the other compounds, can form the basis for a much needed endophenotype for the diagnosis, monitoring and treatment of TBI patients.

The compounds exo-brevicomin (DHB), o-toluidine, α-farnesene, dimethyl sulfone and formanilide could discriminate injured from sham over the time period from 2-14 days post injury (Kimball, B.A. (2016) Bioanalysis 8:1987-1991). Only the first two of these compounds were significantly elevated at 1 hour post-injury in the current study (FIG. 1), emphasizing the relevance of a broad range of clinically relevant times. Data at these earlier time points is particularly relevant for the translation of the mouse results to human studies, as it has been estimated that pathological changes related to TBI might occur anywhere from 5-100 times faster in mice than in humans, and that in the early post-injury period rodent hours might be more equivalent to human days (Agoston, D.V. (2017) J. Neurotrauma 34 (S1): S44-S52; Agoston, et al. (2017) Brain Inj., 31 (9): 1195-1203).

The early post-injury time period is dynamic-brain metabolism, for instance, shifts from hypermetabolic to hypometabolic and neuroinflammation may progress from restorative and beneficial to prolonged and deleterious. The dynamic post-injury environment, and the need to time therapeutic interventions accordingly, has been proposed as one of the reasons for the repeated failure to translate pre-clinical treatments to the human population (Agoston, D.V. (2017) J. Neurotrauma 34 (S1): S44-S52). Biomarkers sensitive to such dynamic changes can greatly improve the ability to diagnose, monitor and treat TBI. Non-invasive body fluids are rich sources of biomarkers and can be collected quickly and easily at multiple time points pre- and post-injury.

The utility of biofluid metabolites is supported by the results here, as nine of the ten compounds identified already have basis in metabolism, evidencing they are robust TBI biomarkers. In particular, 7 of the 10 compounds in the current study can be linked to changes in lipid metabolism. Membrane lipids are released from damaged membranes and their concentration in CSF can increase 10-fold following TBI (Birnic, et al. (2013) BMC Genomics, 14:303), with higher levels being correlated with worse outcomes (Pilitsis, et al. (2003) Neurosci. Lett., 349 (2): 136-8). The released lipids may be peroxidized by reactive oxygen species (ROS) (Kenny, et al. (2019) Crit. Care Med., 47:410-418; Zheng et al., 2014; Yin, et al. (2011) Chem. Rev., 111 (10): 5944-5972), channeled into beta-oxidation to meet energy demands (Prins, M.L. (2008) J. Cereb. Blood Flow Metab. 28:1-16), or metabolized to inflammation modulating compounds by Cytochrome P450 enzymes (Sarapast et al., 2021). Links to all three of these pathways are present in the experimental results.

Five of the ten metabolites observed to increase after brain injury were ketones, with the novel metabolite 3-methyl-4-hexen-2-one showing the clearest increase after TBI. This increase in ketones could be driven by an increase in membrane lipid peroxidation triggered by lipid release from damaged cells along with a sharp increase in ROS (Kenny, et al. (2019) Crit. Care Med., 47:410-418; Zheng et al., 2014). Membrane lipid peroxidation and cleavage produces a wide range of unsaturated, variably oxygenated, primarily straight chain compounds (Kagan, et al. (2017) Nat Chem Biol., 13 (1): 81-90), and these molecules increase sharply after TBI (Kenny, et al. (2019) Crit. Care Med., 47:410-418). Molecules 6-9 carbons in length are common peroxidation products, and all of the non-aromatic ketones observed here were 7-8 carbons in length in addition to being unsaturated and oxygenated.

Volatile ketones can also be produced through an exchange of metabolites between a host and bacteria in the host's gut (Scheline, et al. (1973) Pharmacol. Rev., 25:451-523; Holland, et al. (1983) Life Sci., 32:787-794; Cabrera-Mulero et al. (2019) Rev. Endocr. Metab. Disord., 20 (4): 415-425). TBI in rats produces significant changes in the gut microbiome within hours of injury resulting in loss of microbiota diversity and dysbiosis (Zhu, et al. (2018) Brain Sci., 8 (6): 113). Such alterations are likely to disrupt microbiota metabolism and result in alterations of the volatile metabolome (Kimball, B.A. (2016) Bioanalysis 8:1987-1991). In the current study, the injury dependent increase in the ketone 5-heptene-2-one, seems likely to depend at least in part on bacterial metabolism, as levels of this ketone are lower in the urine of germ-free rats, compared to normal-bacteria control rats (Holland, et al. (1983) Life Sci., 32:787-794).

Phenylacetone was the only aromatic ketone, and its increase may be due to an increase in flux through the aminotransferase pathway for the catabolismof phenylalanine. The post-injury increase in alpha-ketoglutarate (Zheng, et al. (2017) PLOS ONE 12 (8): e0182025) and shortage of reducing equivalents would favor the aminotransferase pathway, the products of which are excreted instead of being metabolized in the TCA cycle, and include the phenylacetone precursor phenylpyruvate.

Two of the significantly elevated compounds, 6-methyl-3-heptanone and DHB, are mouse pheromones (Schaefer, et al. (2010) Chem. Senses 35 (6): 459-471; Fujita, et al. (2020) PLOS One 15 (2): c0229269; Novotny, M.V. (2003) Biochem. Soc. Trans., 31:117-22). The former has the characteristics noted above for lipid peroxidation products and has also been detected in human urine (Smith, et al. (2008) J. Breath Res. 2:037022; Zlatkis, et al. (1981) Clin. Chem., 27:789-97; Drabiska, et al. (2021) J. Breath Res., 15:034001). The latter has not been detected in humans (Drabiska, et al. (2021) J. Breath Res., 15:034001), but its increase may nonetheless be TBI-related. DHB biosynthesis in insects (no vertebrate data is available) involves two highly conserved reactions: β-oxidation of a monounsaturated olefin fatty acid, and epoxidation mediated by a Cytochrome P450 enzyme (Song, et al. (2014) J. Chem. Ecol., 40:181-189). After injury, monounsaturated olefin fatty acids increase (Hogan, et al. (2018) J. Proteome Res., 17 (6): 2131-2143) as does the β-oxidation of fatty acids (Zheng, et al. (2017) PLOS ONE 12 (8): c0182025; Rippce, et al. (2020) Front. Nutr., 7:160; Prins, M.L. (2008) J. Cereb. Blood Flow Metab. 28:1-16; Greco, et al. (2020) Exp. Neurol., 329:113289). Olefin epoxidating Cytochrome P450 enzyme Cyp2el (Meunier, et al. (2004) Chem. Rev., 104:3947-3980) is also upregulated (Birnic, et al. (2013) BMC Genomics, 14:303), and the combination of all these increases may drive the elevation in DHB after injury.

The increase observed in 2-acetyl-2-thiazoline may be due to altered glucose metabolism after TBI. 2-Acetyl-2-thiazoline can be synthesized non-enzymatically by combining cysteamine and methylglyoxal (Ratner, et al. (1937) J. Am. Chem. Soc. 59:200; Schubert, M.P. (1935) J. Biol. Chem., 111:671; Schubert, M.P. (1936) J. Biol. Chem., 114:341-350; Hoffman, et al. (1995) J. Agric. Food Chem., 43, (11): 2946-2950). Cysteamine is produced endogenously from pantothenic acid and present at micromolar levels in brain (Pau, et al. (2019) Front. Neurol., 10:1315). Methylglyoxal is spontaneously formed from glyceraldehyde 3-phosphate, which is the last product formed during glycolysis that does not require cytosolic NAD+. Cytosolic NAD+ is severely depleted after injury which would be expected to increase both glyceraldehyde 3-phosphate and methylglyoxal. In addition, a recent metabolomics study of TBI patients found an increase in the methylglyoxal pathway after injury (Hagos, et al. (2018) Crit Care Med., 46 (9): 1471-1479), all of which could account for the increase observed in 2-acetyl-2-thiazoline. Of note, increased flux through the methylglyoxal pathway, which does not require cytosolic NAD+, would also allow the injured brain to continue supplying pyruvate to the mitochondria for ATP production at a time when energy demand is high and yet normal glycolysis is severely compromised.

Two compounds of exogenous origin were also significantly elevated in the samples and may have been affected by TBI. o-Toluidine is an environmental toxin for which detoxification and routing to the urine depends on the enzymes Cyp1Al and Cyp 2E1, both of which are transiently upregulated several-fold after TBI (Birnie, et al. (2013) BMC Genomics, 14:303), possibly accounting for the increased o-toluidine observed herein. Another exogenous compound detected in this study, and whose solubility may also have increased after injury, is a derivative of the widely used antioxidant Irganox® 1076, a molecule designed to interact with and be oxidized by reactive oxygen species. The oxidized form is enormously more water soluble than the parent compound, and the sharp increase in ROS after TBI may increase the oxidation and solubility of this antioxidant, and account for its increased concentration in urine after TBI.

In summary, nine of the ten compounds identified already have links to TBI pathophysiology, indicating the markers are reliable markers for TBI. The volatile ketones altered in the first few hours following injury are prime targets for diagnosing TBI. In particular, the novel metabolite 3-methyl-4-hexen-2-one is an excellent biomarker candidate for early diagnosis. Although the precise details may be different between mice and humans (e.g., time course of metabolic changes), the underlying mechanisms are the same (including increases in reactive oxygen species, fatty acid β-oxidation and membrane lipid peroxidation, as well as perturbations in cerebral energy metabolism and gut microbiota), and the methodology applied here to mouse urine sample could easily be extended to human urine samples. There is a profound need for TBI biomarkers, particularly at early time points. The present study provides such biomarkers, thereby improving the diagnosis, monitoring and treatment of TBI.

### EXAMPLE 2

As described herein, urine biomarkers of brain injury are provided which are aliphatic ketones having four to ten carbons. In certain embodiments, the aliphatic ketone comprises a ketone on carbon number two. To confirm the results observed in a mouse model of traumatic brain injury, the study was extended to human subjects.

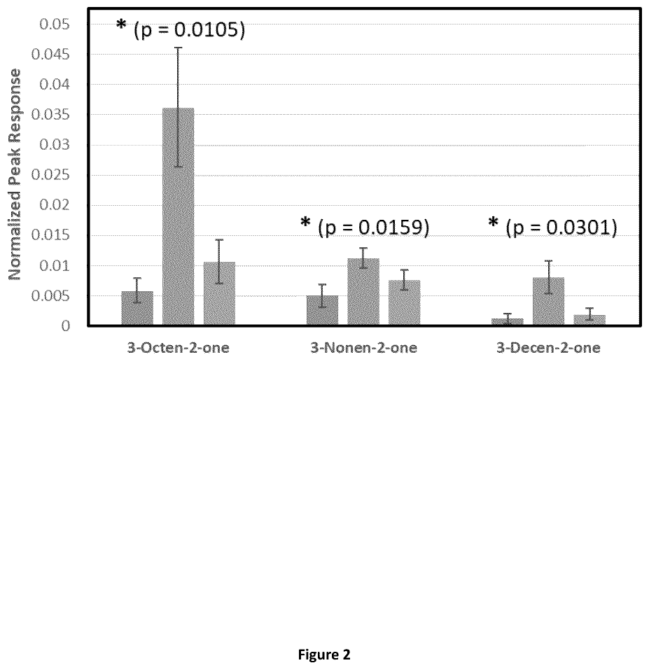

Briefly, urine samples were collected from concussed youth and healthy controls. Samples from the concussed youth were obtained either on the day of the injury or one day post injury. Samples were analyzed for the presence of ketones, specifically 3-octen-2-one, 3-nonen-2-one, and 3-decen-2-one. As seen in FIG. 2, significant increases in urine of the three ketones with eight, nine, or ten carbons was observed soon after injury. Notably, the increase in the ketone was short lived as levels of the ketones returned to near normal (pre-injury) levels within about a day. These results provide further evidence that urinary aliphatic ketones serve as early biomarkers for brain injury in humans.

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for detecting and/or diagnosing a traumatic brain injury in a subject comprising detecting and/or measuring at least one biomarker in a biological sample obtained from said subject.

2. The method of claim 1, wherein said biological sample is a fluid.

3. The method of claim 2, wherein said biological fluid is urine.

4. The method of claim 1, wherein the biomarkers are detected and/or measured in the gaseous or vapor phase of the biological sample.

5. The method of claim 1, wherein said traumatic brain injury is mild or moderate.

6. The method of claim 1, wherein said traumatic brain injury is a concussion.

7. The method of claim 1, further comprising obtaining said biological sample from said subject.

8. The method of claim 1, wherein at least one biomarkers is an aliphatic ketone.

9. The method of claim 8, wherein said aliphatic ketone comprises a ketone group at position 2.

10. The method of claim 9, wherein said aliphatic ketone further comprises a double bond between carbons 3 and 4.

11. The method of claim 10, wherein said aliphatic ketone has four to twelve carbons.

12. The method of claim 1, wherein said at least one biomarker comprises 3-octen-2-one, 3-nonen-2-one, and/or 3-decen-2-one.

13. The method of claim 1, wherein said at least one biomarker comprises at least one of phenylacetone, 3-methyl-4-hexen-2-one, 4-hepten-2-one, 5-hepten-2-one, antioxidant derivative 1, 2-acetyl-2-thiazole, o-Toluidine, 2,3-dehydro-exo-brevicomin (DHB), butyl ether, and 6-methyl-3-heptanone.

14. The method of claim 13, wherein said at least one biomarker comprises 3-methyl-4-hexen-2-one and/or phenylacetone.

15. The method of claim 1, wherein said detecting and/or measuring of at least one biomarker comprises gas chromatography, mass spectrometry, and/or ion mobility spectrometry.

16. The method of claim 1, wherein said at least one biomarker is measured and/or detected by a field, portable, and/or handheld device.

17. The method of claim 1, wherein said biological sample is obtained from the subject within 48 hours of a purported traumatic brain injury.

18. The method of claim 1, wherein said biological sample is obtained from the subject within 24 hours of a purported traumatic brain injury.

19. The method of claim 1, wherein said biological sample is obtained from the subject within 12 hours of a purported traumatic brain injury.

20. The method of claim 1, wherein said biological sample is obtained from the subject within 6 hours of a purported traumatic brain injury.

21. The method of claim 1, wherein said biological sample is obtained from the subject within 1 hour of a purported traumatic brain injury.

22. The method of claim 1, wherein the presence and/or increase in the at least one biomarker indicates the subject has a traumatic brain injury.

23. The method of claim 1, further comprising administering a traumatic brain injury therapeutic to the subject determined to have a traumatic brain injury.

24. The method of claim 23, wherein said traumatic brain injury therapeutic comprises a branched chain amino acid.

25. A device for detecting and/or diagnosing a traumatic brain injury in a subject, said device configured to detect and/or measure at least one biomarker in a biological sample obtained from said subject.

26. The device of claim 25, which is field, portable, and/or handheld device.

27. The device of claim 25, wherein said device is configured to detect and/or measure at least one biomarker in a fluid and/or gas.

28. The device of claim 25, wherein said device comprises a gas chromatographer, mass spectrometer, and/or ion mobility spectrometry.
